(19) United States
(12) Patent Application Publication
(10) Pub. No.: US 2024/0366860 A1
(43) Pub. Date: Nov. 7, 2024

(54) PUMP FLOW OPTIMIZATION

(71) Applicant: CareFusion 303, Inc., San Diego, CA (US)

(72) Inventor: Raymond P. FEITH, Chino Hills, CA (US)

(21) Appl. No.: 18/683,992

(22) PCT Filed: Aug. 30, 2022
(86) PCT No.: PCT/US2022/042081

§ 371 Date: Feb. 15, 2024

Related U.S. Application Data
(60) Provisional application No. 63/239,330, filed on Aug. 31, 2021.

(51) Int. Cl. A61M 5/142; A61M 5/14

(52) U.S. Cl. CPC A61M 5/14212; A61M 5/1407; A61M 2005/14208; A61M 2205/0216; A61M 2205/3334; A61M 2205/502; A61M 2205/60

(57) ABSTRACT

A medical device includes a body configured to receive and secure an IV tube that is configured to allow for the transportation of a fluid. The medical device includes a pump mechanism including a first element positioned along a first axis and configured to move in a first direction along the first axis to compress the IV tube. The pump mechanism includes a second element positioned along a second axis perpendicular to the first axis and configured to move in a second direction along the second axis and perpendicular to the first direction to provide a force against a side of the IV tube. The pump mechanism includes a third element positioned along the second axis opposite the second element and configured to move in a third direction along the second axis opposite the second direction to provide a force against another side of the IV tube.

### BACKGROUND

The present disclosure generally relates to an intravenous (IV) set or infusion pump flow control, and in particular one or more pump elements to rebound an IV tube.

### SUMMARY

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. Often, the flow rate through the tubing is controlled via a pumping mechanism. The pump mechanism compresses a portion of the IV set such that fluid travels within the IV set. The pump mechanism continuously compresses the IV set to achieve a desired flow rate. However, existing pump mechanisms can be inaccurate and provide inconsistent flow rates due to the rate at which they compress the IV set. Existing pump mechanisms may repeatedly compress IV sets without allowing the IV sets to return to their original states. As such, the existing pump mechanisms can compress an IV set with varying shapes between compression strokes which may result in inaccurate or inconsistent flow rates or volumes.

Thus, it is desirable to provide a pump mechanism that improves the reliability of flow rates and volumes.

Various implementations of systems, methods, and devices within the scope of the appended claims each have several aspects, no single one of which is solely responsible for the attributes described herein. Without limiting the scope of the appended claims, after considering this disclosure, and particularly after considering the section entitled “Detailed Description” one will understand how the aspects of some implementations are used to provide accurate and consistent flow rates. One or more implementations provide a pump mechanism including a first element positioned along a first axis, a second element positioned along a second axis perpendicular to the first axis, and a third element positioned along the second axis opposite the second element. The first element of the pump mechanism is configured to compress an IV tube of the cause to fluid to flow through the IV tube, and the second and third elements are configured to apply separate forces at opposite sides of the IV tube to cause the IV tube to return to its original (uncompressed) state. The pump mechanism is included in a medical device, such as an infusion pump, including one or more features, all as disclosed in the drawings and specification.

In one aspect, a medical device includes a body configured to receive and secure an IV tube in an extended position. The IV tube is configured to allow for the transportation of a fluid. The medical device also includes a pump mechanism residing within the body. The pump mechanism includes a first element positioned along a first axis and configured to, when actuated, move in a first direction along the first axis to compress the IV tube when the IV tube is received and secured in the body. The pump mechanism also includes a second element positioned along a second axis perpendicular to the first axis and configured to, when actuated, move in a second direction along the second axis and perpendicular to the first direction to provide a first force against a first side of the IV tube. The pump mechanism further includes a third element positioned along the second axis opposite the second element and configured to, when actuated, move in a third direction along the second axis opposite the second direction to provide a second force against a second side of the IV tube, the second force opposing the first force.

In some implementations, the medical device further includes a processor configured to cause the first element to be actuated at a first time compressing the IV tube and causing the fluid to flow, and cause the second and third elements to be contemporaneously actuated at a second time distinct from the first time. At the second time, the second element provides to the first force against the first side of the IV tube and the third element provides the second force against the second side of the IV tube and cause the IV tube to rebound to a resting state.

In some implementations, the first, second, and third elements are synchronized such that the first, second, and third elements are actuated in unison with the second and third elements being actuated in directions opposite to the second and third directions, respectively, at the first time; and the first element being actuated in a direction opposite the first direction at the second time. In some implementations, the first element is actuated a third time following the second time only after the IV tube is rebound to a resting configuration. In some implementations, the first element comprises a first plurality of members, and each member of the first plurality of members is moved sequentially from a first end to a second end. In some implementations, the second element comprises a second plurality of members and the third element comprises a third plurality of members. Each member of the second plurality of members is moved sequentially from a first end to a second end, and each member of the third plurality of members is moved sequentially from a first end to a second end.

In some implementations, the pump mechanism is a first pump mechanism and the medical device further includes a second pump mechanism. The first element is actuated by the first pump mechanism and the second and third elements are actuated by the second pump mechanism. In some implementations, the body comprises a door and a housing. The door is configured to couple to the housing when closed. In some implementations, the first pump mechanism is located within a part of the housing, and the second pump mechanism is located within a portion of the door.

In some implementations of the medical device, the first element may include a first surface that compresses the IV tube, and the second and third elements have a second surface configured to reshape the IV tube. In some implementations, the first surface is a flat surface configured to compresses the IV tube flat against a surface of the body, and the second surface is a concave surface configured to reshape the IV tube. In some implementations, the second surface includes one or more actuators, and the second surface dynamically adjusts to conform with the IV tube being used. In some implementations, the second surface includes deformable material, and the second surface dynamically adjusts to conform with the IV tube being used. In some implementations, the processor of the medical device, upon receiving the IV tube, detect an identifier associated with the IV tube. The identifier includes one or more of an IV tube type, an IV tube diameter, an IV tube wall thickness, IV tube shape, IV tube pliability, IV tube flexibility, and an IV tube material. The processor, based on the identifier, adjusts the contour of the second surface, via the one or more actuators, such as to change the shape of the second surface to substantially mate with a portion of the IV tube. Alternatively, when the second surface includes deformable material, the processor, based on the identifier, adjusts the contour of the second surface, via first and second forces applied by the second and third elements, respectively, such as to change the shape of the second surface to substantially mate with a portion of the IV tube.

In some implementations of the medical device, the processor is further configured to upon receiving the IV tube, detect an identifier associated with the IV tube. The identifier includes one or more of an IV tube type, an IV tube diameter, an IV tube wall thickness, IV tube shape, IV tube pliability, IV tube flexibility, and an IV tube material. The processor is further configured to, based on the identifier, determine a compression force to compress the IV tube, the first force to be applied against the first side of the IV tube, and the second force to be applied against the second side of the IV tube; and generate the compression force, the first force, and the second force via actuation of the first, second, and third elements, respectively.

In another aspect, a method of controlling a flow rate is provided. The method includes at a pump mechanism of a medical device, the pump mechanism including a first element positioned along a first axis, a second element positioned along a second axis perpendicular to the first axis, and a third element positioned along the second axis opposite the second element. The method includes actuating the first element in a first direction along the first axis, wherein the first element is configured to compress an intravenous (IV) tube received and secured in a body of the medical device. The method also includes actuating the second element in a second direction along the second axis and perpendicular to the first direction, wherein the second element is configured to provide a first force against a first side of the IV tube. The method further includes actuating the third element in a third direction along the second axis opposite the second direction to provide a second force against a second side of the IV tube, the second force opposing the first force.

In some implementations, the method further includes causing the first element to be actuated at a first time compressing the IV tube and causing the fluid to flow, and causing the second and third elements to be contemporaneously actuated at a second time distinct from the first time. At the second time, the second element provides to the first force against the first side of the IV tube and the third element provides the second force against the second side of the IV tube and cause the IV tube to rebound to a resting state.

In some implementations, the method further includes upon receiving the IV tube, detecting an identifier associated with the IV tube. The identifier includes one or more of an IV tube type, an IV tube diameter, an IV tube wall thickness, and an IV tube material. The method further includes, based on the identifier, determining a compression force to compress the IV tube, the first force to be applied against the first side of the IV tube, and the second force to be applied against the second side of the IV tube; and generating the compression force, the first force, and the second force via actuation of the first, second, and third elements, respectively. In some implementations, the method further includes based on the identifier, adjusting the contour of a second surface of the second and third elements, via a shaping means, such as to change the shape of the second surface to substantially mate with a portion of the IV tube. In some implementations, the shaping means includes one or more actuators and/or deformable material.

The foregoing and other features, aspects and advantages of the disclosed implementations will become more apparent from the following detailed description and accompanying drawings.

### BRIEF DESCRIPTION OF THE DRAWINGS

The accompanying drawings, which are included to provide further understanding of the disclosure and are incorporated in and constitute a part of this specification, illustrate implementations of the disclosure and together with the description serve to explain the principles of the disclosure.

FIG. 1 shows a patient care system, in accordance with various aspects of the present disclosure.

FIG. 2 shows an infusion pump, in accordance with various aspects of the present disclosure.

FIGS. 3A-3C illustrate a prior art example of a pump mechanism and its operational use.

FIGS. 4A and 4B illustrate operational use of a pump mechanism residing within a body of an infusion pump, in accordance with some implementations.

FIGS. 5A-5C illustrate operation use of a pump mechanism, in accordance with some implementations.

FIG. 6 illustrates another implementation of a pump mechanism in accordance with some implementations.

FIG. 7 illustrates a flow diagram of a method for controlling a flow rate of a medical device, in accordance with some implementations.

FIG. 8 is a conceptual diagram illustrating an example electronic system for controlling a flow rate of a medical device, according to aspects of the subject technology.

Like reference numerals refer to corresponding parts throughout the drawings.

### DETAILED DESCRIPTION

The disclosed pump mechanism incorporates additional pump elements that return an IV set to its original (uncompressed) state after it has been compressed. The additional pump elements are synchronized with compression members of the pump mechanism to ensure that the IV set is in its original state before the IV set is compressed. The pump mechanism prevents the IV set from being compressed before it returns to its original state thereby improving the accuracy and consistency of the flow rate generated by the pump mechanism.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to controlling and maintaining a flow rate during the administration of medical fluid using the disclosed pump mechanism, it is to be understood that this description is only an example of usage and does not limit the scope of the claims. Various aspects of the disclosed pump mechanism may be used in any application where it is desirable to control a fluid flow rate.

The disclosed pump mechanisms overcome several challenges discovered with respect to certain conventional pump mechanisms. One challenge with certain conventional pump mechanisms is that the accuracy and consistency of the flow rate can change with time and/or based on the selected flow rate. Because the inaccuracies or the inconsistencies of pump mechanisms can alter the flow rate of the administered medical fluid, monitoring the pump and ensuring the efficacy of a treatment provided by the pump can be difficult.

Therefore, in accordance with the present disclosure, it is advantageous to provide a pump mechanism as described herein that eliminates or substantially reduces the inaccuracy and inconsistency in the generated flow rates. The disclosed pump mechanism provides additional pump elements that enable the IV set to rebound before it is compressed thereby improving the accuracy and consistency of the generated flow rate.

An example of a pump mechanism that improves the accuracy and consistency of the generated flow rates is now described.

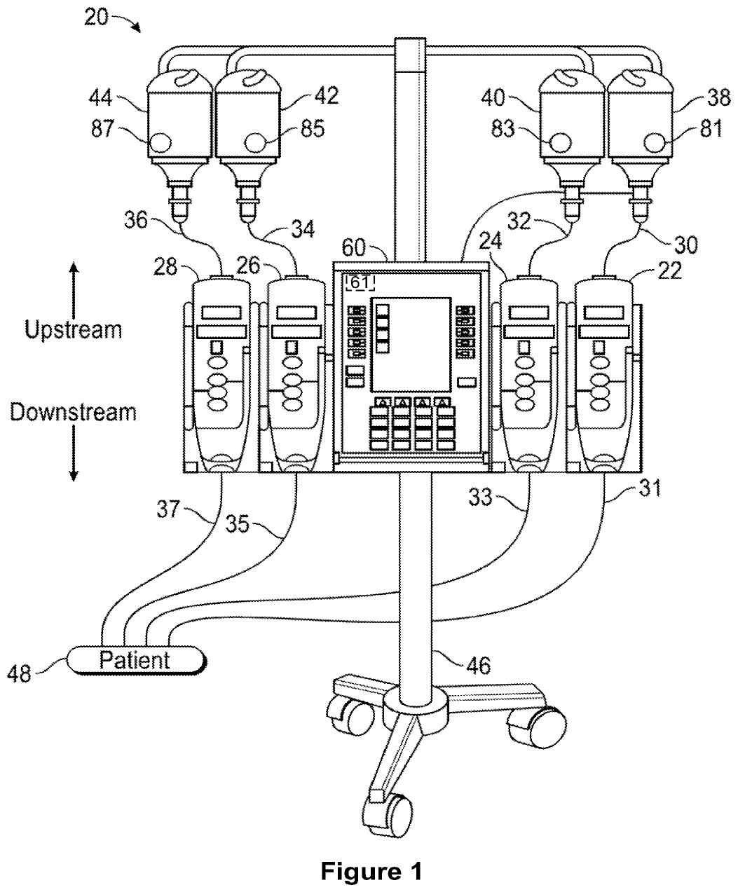

FIG. 1 shows a patient care system, in accordance with various aspects of the present disclosure. The patient care system 20 includes four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, tabletop, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a controller 60. In some implementations, the controller 60 is communicatively coupled to memory 61 storing one or more instructions for operating the infusion pumps 22, 24, 26, and 28 and/or other collected data as described below.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typically, medical fluid administration sets have more parts than are shown in FIG. 1. Many have check valves, drip chambers, valves with injection ports, connectors, and other devices well known to those skilled in the art. These other devices have not been included in the drawings so as to preserve clarity of illustration.

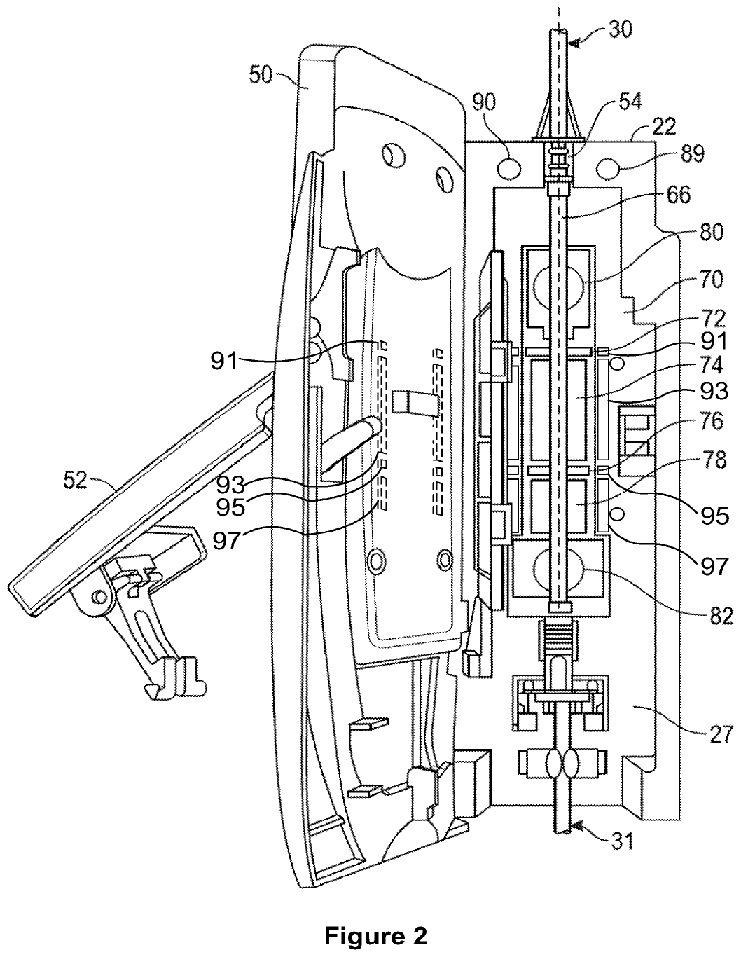

Turning now to FIG. 2, an infusion pump 22 having a body 27 is shown in perspective view, in accordance with various aspects of the present disclosure. The infusion pump 22 is shown with the front door 50 open, showing the upstream fluid line 30 and downstream fluid line 31 in operative engagement with the pump 22. The infusion pump 22 directly acts on a tube 66 that connects the upstream fluid line 30 to the downstream fluid line 31 to form a continuous fluid conduit, extending from the respective fluid supply 38 (FIG. 1) to the patient 48, through which fluid is acted upon by the pump to move fluid downstream to the patient. Specifically, a pumping mechanism 70 acts as the flow control device of the pump to move fluid though the conduit. The upstream and downstream fluid lines and/or tube 66 may be coupled to a pump cassette or cartridge that is configured to be coupled to the pump 22.

The type of pumping mechanism may vary and may be for example, a multiple finger pumping mechanism. For example, the pumping mechanism may be of the “four finger” type and includes an upstream occluding finger 72, a primary pumping finger 74, a downstream occluding finger 76, and a secondary pumping finger 78. The “four finger” pumping mechanism and mechanisms used in other linear peristaltic pumps operate by sequentially pressing on a segment of the fluid conduit by means of the cam-following pumping fingers and valve fingers 72, 74, 76, and 78. The pressure is applied in sequential locations of the conduit, beginning at the upstream end of the pumping mechanism and working toward the downstream end. At least one finger is always pressing hard enough to occlude the conduit. As a practical matter, one finger does not retract from occluding the tubing until the next one in sequence has already occluded the tubing; thus, at no time is there a direct fluid path from the fluid supply to the patient. The operation of peristaltic pumps including four finger pumps is well known to those skilled in the art and no further operational details are provided here.

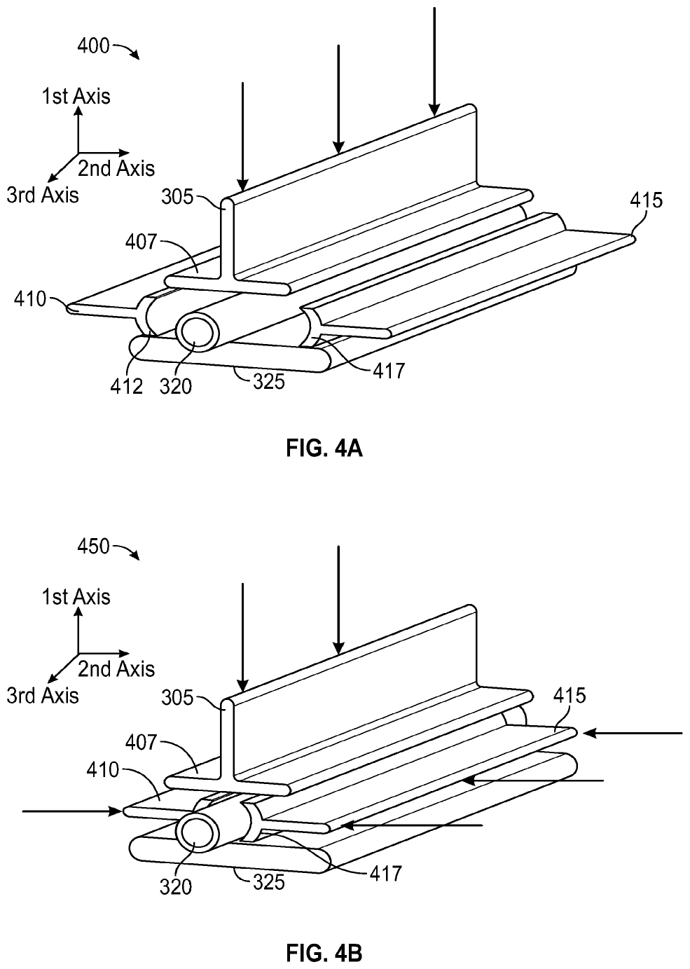

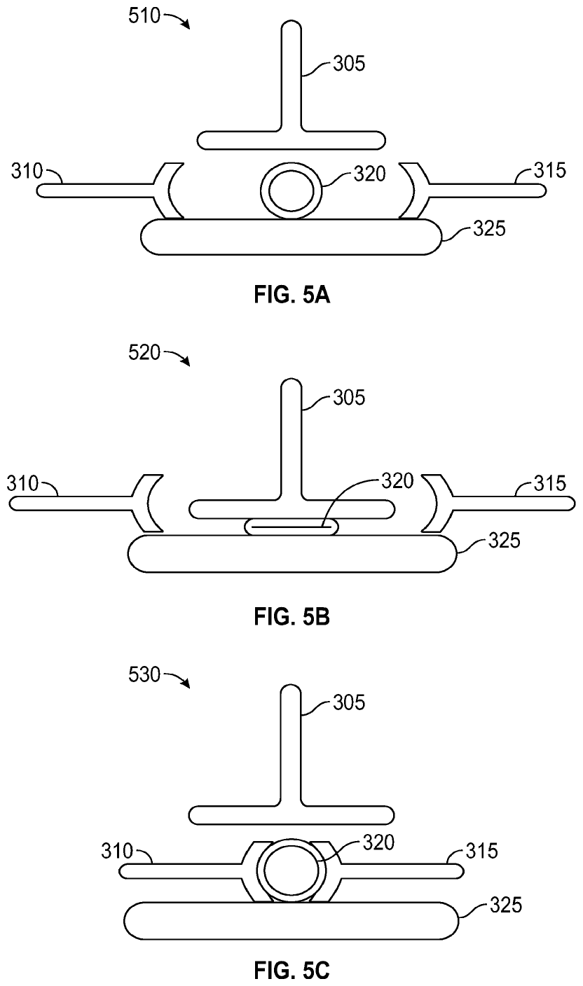

In some implementations, the pumping mechanism includes additional pump elements 91, 93, 95, and 97 on each side of the pumping fingers and valve fingers 72, 74, 76, and 78. In some implementations, the additional pump elements 91, 93, 95, and 97 are used to rebound tube 66 after it has been compressed. Additional detail on rebounding tube 66 is provided below in reference to FIGS. 4A-5C. In some implementations, the additional pump elements 91, 93, 95, and 97 are coupled to the same cam as the pumping fingers and valve fingers 72, 74, 76, and 78. Alternatively, in some implementations, the additional pump elements 91, 93, 95, and 97 are coupled to a separate cam that the one coupled to the pumping fingers and valve fingers 72, 74, 76, and 78. In some implementations, the additional pump elements 91, 93, 95, and 97 are part of the same pump mechanism as the pumping fingers and valve fingers 72, 74, 76, and 78. Alternatively, in some implementations, the additional pump elements 91, 93, 95, and 97 are part of a distinct pump mechanism. In some implementations, the additional pump elements 91, 93, 95, and 97 are part of, or coupled to body 27. Alternatively, in some implementations, the additional pump elements 91, 93, 95, and 97 are part of, or coupled to door 50 (as represented by the dotted lines for the additional pump elements 91, 93, 95, and 97).

FIG. 2 further shows a downstream pressure sensor 82 included in the pump 22 at a downstream location with respect to the pumping mechanism. The downstream pressure sensor 82 is mounted to the flow control device 70 and is located adjacent and downstream in relation to the flow control device. The downstream pressure sensor is located downstream from the flow control device, that is, at a location between the patient 48 (FIG. 1) and the flow control device, so that the connection of the correct fluid supply with the correct pump may be verified before any fluid is pumped to the patient.

With reference still to FIG. 2, an upstream pressure sensor 80 may also be included in the pump 22. The upstream pressure sensor is assigned to the flow control device or pumping mechanism 70 and, in this implementation, is further provided as an integral part of the pump 22. It is mounted to the flow control device 70 and is located adjacent and upstream in relation to the flow control device. The upstream pressure sensor is located upstream from the flow control device, that is, at a location between the fluid supply 38 (FIG. 1) and the flow control device, so that the connection of the correct fluid supply with the correct pump may be verified before any fluid is pumped to the patient.

The pump 22 or a portion of the pump 22 may also be equipped with an electronic data tag or data transmitter. For example, as shown in FIG. 2, pump 22 may be equipped with a data tag 89 or a reader device 90 for providing or receiving infusion data. The data reader devices may include RFID readers (or receivers) or other wireless devices that are compatible with the data tags associated with the fluid containers. A data transmitter may transmit interrogation signals to the electronic data tags 81, 83, 85, 87 associated with the fluid containers for obtaining infusion data from those tags. Although referred to as data transmitting devices or RFID tags or RFID transponders, data transmitting devices may also receive or read data and may also be writable.

Typically, medical tubing is a disposable product that is used once and then discarded. The medical tubing may be formed from any suitable material, e.g., soft PVC, silicone, thermoplastic vulcanizate (TPV) (ethylene propylene diene monomer (EPDM)+polypropylene (PP)), thermoplastic polyurethane (TPU), thermoplastic styrenic elastomer (TPS) (styrene-butadiene-styrene (SBS)/styrene-ethylene-butylene-styrene (SEBS)/styrene-isoprene-rubber (SIS)/styrene-ethylene-propylene-styrene (SEPS)) and its blending with polyolefin, thermoplastic polyester elastomer (TPEE) (polyether ester) rubber). As shown in FIG. 2, medical tubing 66 may be inserted into or otherwise engaged by pump 22. Pump 22 may include any of Large Volume, patient-controlled analgesia (PCA), ambulatory pump or insulin pump that drive tubing segment(s) to deliver medication or nutrients into a patient's body in controlled amounts. The medical tubing 66 is compressed when the pump door 50 is closed. With the pump door 50 closed, the medical tubing 66 is constrained within a gap 54 and directly contacted by the upstream force sensor 80. As discussed above, there are many sources of variation in measuring the force on the medical tubing 66 by the sensor 80.

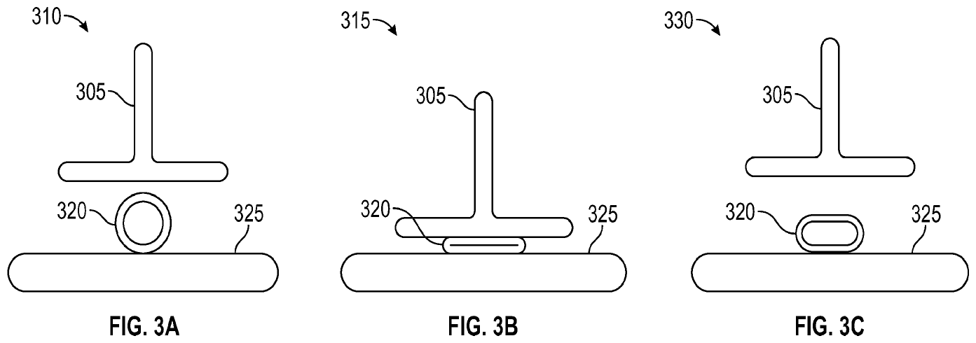

FIGS. 3A-3C illustrate a prior art example of a pump mechanism and its operational use. A first view 310 shows a pump mechanism including a first element 305. The first element 305 is configured to compress an IV tube 320 and cause fluid to flow through the IV tube 320. For example, as shown in second view 315, the first element 305 compresses the IV tube 320 on a surface 325 of the body of a medical device (such as infusion pump 22). As further shown in the third view 330, once the IV tube 320 is no longer compressed (i.e., the first element 305 is moved away) the IV tube 320 starts to move back to its original (uncompressed) state. However, in many instances, the IV tube 320 is not able to return to its original state before the first element 305 compresses the IV tube 320 a second time. Because the IV tube 320 is not able to return to its original state before the first element 305 compresses the IV tube 320 a second time, the flow rate generated by the prior art pump mechanism can be inaccurate. In particular, the prior art pump mechanism relies on the IV tube 320 being able to rebound quickly enough before it is compressed again to provide consistent flow rates, which is not always possible. As faster flow rates are targeted by the medical device, less times is allotted for the IV tube 320 to return to its original state before it is compressed a second time. Further, due to variations in tube diameter, wall thickness, and a durometer along with additional mechanical properties; the displaced fluid volume per stroke varies in prior art solutions.

FIGS. 4A and 4B illustrate operational use of a pump mechanism residing within a body of an infusion pump, in accordance with some implementations. As described above in reference to FIG. 2, in some implementations, the pump mechanism is configured to operate as a flow control device of the infusion pump (e.g., infusion pump 22; FIG. 2) to move fluid though the conduit. In some implementations, the infusion pump includes a first element 305, a second element 410, and a third element 415. The first element 305 is an example of fingers 72, 74, 76, and/or 78 described above in reference to FIG. 2. The second element 410 and the third element 415 are examples of the additional pump elements 91, 93, 95, and 97 described above in reference to FIG. 2. The body (e.g., body 27; FIG. 2) is configured to receive and secure an IV tube 320 in an extended position, and the IV tube 320 is configured to allow for the transportation of a fluid. The IV tube 320 is an instance of tube 66 described above in reference to FIG. 2.

In some implementations, the first element 305 is positioned along a first axis and configured to, when actuated, move in a first direction along the first axis to compress an IV tube 320 when the IV tube 320 is received and secured in the body. For example, as shown in first operational view 400, the first element 305 moves along the 1st axis and compresses the IV tube 320. In some implementations, the first element 305 has a first surface that compresses the IV tube 320. In some implementations, the first surface is a flat surface 407 configured to compresses the IV tube flat 320 against a surface 325 of the body. In some implementations, surface 325 of the body is a portion of a door 50 (FIG. 2). In some implementations, the first element 305 is coupled with a cam and moves according to the movement of the cam. Additional information on the movement of the first element 305 is provided above in reference to FIG. 2.

The second element 410 is positioned along a second axis perpendicular to the first axis and configured to, when actuated, move in a second direction along the second axis and perpendicular to the first direction to provide a first force against a first side of the IV tube 320. The third element 415 is positioned along the second axis opposite the second element and configured to, when actuated, move in a third direction along the second axis opposite the second direction to provide a second force against a second side of the IV tube 320, the second force opposing the first force. For example, as shown in second operational view 450, the second element 410 moves in the second direction along the second axis and the third element 415 moves in an opposite direction. In some implementations, the second and third elements 410 and 415 have a second surface configured to reshape the IV tube 320. In some implementations, the second surface is a concave shape 417. Alternatively, in some implementations, the second surface is a curved surface, a clamp, or other shape to substantially engage with a wall of the tube to convey a force sufficient to return the tube to its resting (e.g., uncompressed) diameter. In some implementations, the second surface is shaped based on the size and shape of the of the IV tube 320. For example, the second surface may include actuators or deformable material that can be dynamically adjusted to conform with the IV tube 320 being used. In such implementations, the fluid pump (e.g., infusion pump 22; FIG. 2) may receive information identifying the IV tube 320. Based on that information, the fluid pump may obtain characteristics of the IV tube 320 such as shape, diameter, pliability, flexibility, etc. The second and third elements or portions thereof (e.g., the second surface) may be adjusted based on the obtained characteristics. For example, the diameter of the IV tube 320 may be used to generate a pushing distance for the second and third elements. The pliability or flexibility may be used to generate a force to be applied by the second and third elements. The shape may be used to adjust the contour of the second surface such as via actuation of a shaping means to change the shape of the second surface.

In some implementations, the infusion pump includes one or more processors (e.g., controller 60; FIG. 1) configured to adjust the contour of the second surface of the second and third elements 410 and 415. In particular, the one or more processors are configured to, upon receiving the IV tube 320, detect an identifier associated with the IV tube 320. The identifier including one or more of an IV tube type, an IV tube diameter, an IV tube wall thickness, IV tube shape, IV tube pliability, IV tube flexibility, and an IV tube material. The one or more processors, based on the identifier, adjust the contour of the second surface such as to change the shape of the second surface to substantially mate with a portion of the IV tube 320. The contour of the second surface can be adjusted via the one or more actuators or, in the case of deformable material, via the respective force to the IV tube 320 applied by the second and third elements 410 and 415. The respective force applied by the second and third elements 410 and 415 can be determined by the identifier associated with the IV tube 320 as discussed below.

In some implementations, the second and third elements 410 and 415 are coupled with the cam coupled to the first element 305 and move according to the movement of the cam. In other words, the first, second, and third elements 305, 410, and 415 can be coupled to and operated by a single cam. Alternatively, in some implementations, the second and third elements 410 and 415 are coupled with an additional cam and move according to the movement of the additional cam. Additional information on the movement of the second and third elements 410 and 415 is provided above in reference to FIG. 2.

In some implementations, the infusion pump includes one or more processors (e.g., controller 60; FIG. 1) configured to operate the pump mechanism. In particular, in some implementations, the one or more processors cause the first element 305 to be actuated at a first time compressing the IV tube 320 and causing the fluid to flow, and cause the second and third elements 410 and 415 to be contemporaneously actuated at a second time distinct from the first time. At the second time, the second element provides to the first force against the first side of the IV tube and the third element provides the second force against the second side of the IV tube 320 and cause the IV tube 320 to rebound to a resting state. In some implementations, the first, second, and third elements 305, 410, and 415 are synchronously actuated. More specifically, the first, second, and third elements 305, 410, and 415 are synchronized such that the first, second, and third elements 305, 410, and 415 are actuated in unison with the second and third elements 410 and 415 being actuated in directions opposite to the second and third directions, respectively, at the first time; and the first element 305 being actuated in a direction opposite the first direction at the second time. The first element is actuated a third time following the second time only after the IV tube 320 is rebound to a resting configuration. More specifically, the first element is configured to compress the IV tube 320 a second time after the IV tube 320 returns to its original shape.

In some implementations, the one or more processors of the infusion pump are configured to, upon receiving the IV tube 302, detect an identifier associated with the IV tube 320. One or more characteristics of the IV tube 320 associated with the identifier are described above. The one or more processors of the infusion pump are configured to, based on the identifier, determine a compression force to compress the IV tube 320, the first force to be applied against the first side of the IV tube 320, and the second force to be applied against the second side of the IV tube 320; and generate the compression force, the first force, and the second force via actuation of the first, second, and third elements 305, 410, and 415, respectively.

FIGS. 5A-5C illustrate operation use of a pump mechanism, in accordance with some implementations. The pump mechanism in FIGS. 5A-5C is an implementation of any pump mechanism described above in reference to FIGS. 1-2 and 4A-4B. A first view 810 shows a pump mechanism including a first element 305, a second element 410, and a third element 415. The first element 305 is configured to compress an IV tube 320 and cause fluid to flow through the IV tube 320 as described above. The second view 520 shows the IV tube 320 compressed against a surface 325 of a body of a medical device (such as infusion pump 22) by the first element 305. The third view 530 shows the second and third elements 410 and 415 providing respective forces against opposite sides of the IV tube 320. As described above, the second and third elements 410 and 415 are configured to rebound the IV tube 320 to its original state. Movement of the first, second, and third elements 305, 410, and 415 is synchronized such that the first element 305 is actuated at first time and the second and third elements 410 and 415 are actuated at a second time distinct from the first time. No additional delay is introduced by the actuation of the second and third elements 410 and 415.

The second and third elements 410 and 415 are configured to rebound the IV tube 320 to its original state before the first element 305 is actuated a second time. This ensures that the pump mechanism provides accurate and consistent flow rates. Additionally, actuation of the second and third elements 410 and 415 to rebound the IV tube 320 allows of the medical device to provide faster flow rates without having to wait for the IV tube 320 return to its original state on its own. Further, the pump mechanism disclosed herein is able to achieve accurate and consistent flow rates regardless of variations in tube diameter, wall thickness, and/or a durometer along with additional mechanical properties.

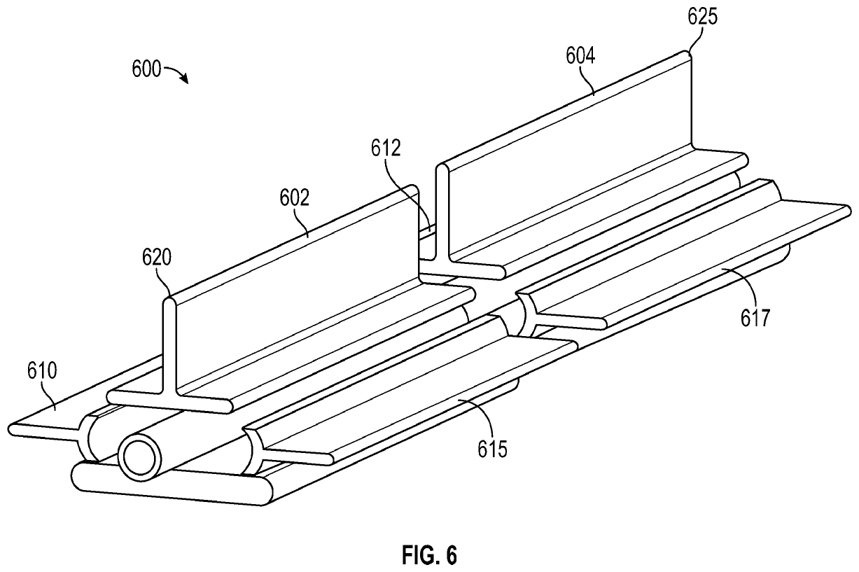

FIG. 6 illustrates another implementation of a pump mechanism in accordance with some implementations. In some implementations, a first element (e.g., first element 305; FIG. 4A-5C) includes a first plurality of members 602 and 604; a second element (e.g., second element 410; FIG. 4A-5C) includes a second plurality of members 610 and 612; and a third element (e.g., third element 415; FIG. 4A-5C) includes a third plurality of members 615 and 617.

As described above in reference to FIG. 2, each member of the first plurality of members 602 and 604 is moved sequentially from a first end 620 to a second end 625. Similarly, each member of the second plurality of members 610 and 612 and each member of the third plurality of members 615 and 617 are moved sequentially from the first end 620 to the second end 625. The additional members allow for improved flow rate control. More specifically, each individual member can be controlled to achieve a desired flow rate. The first plurality of members 602 and 604 is an example of fingers 72, 74, 76, and/or 78 described above in reference to FIG. 2. The second plurality of members 610 and 612 and the third plurality of members 615 and 617 are examples of the additional pump elements 91, 93, 95, and 97 described above in reference to FIG. 2.

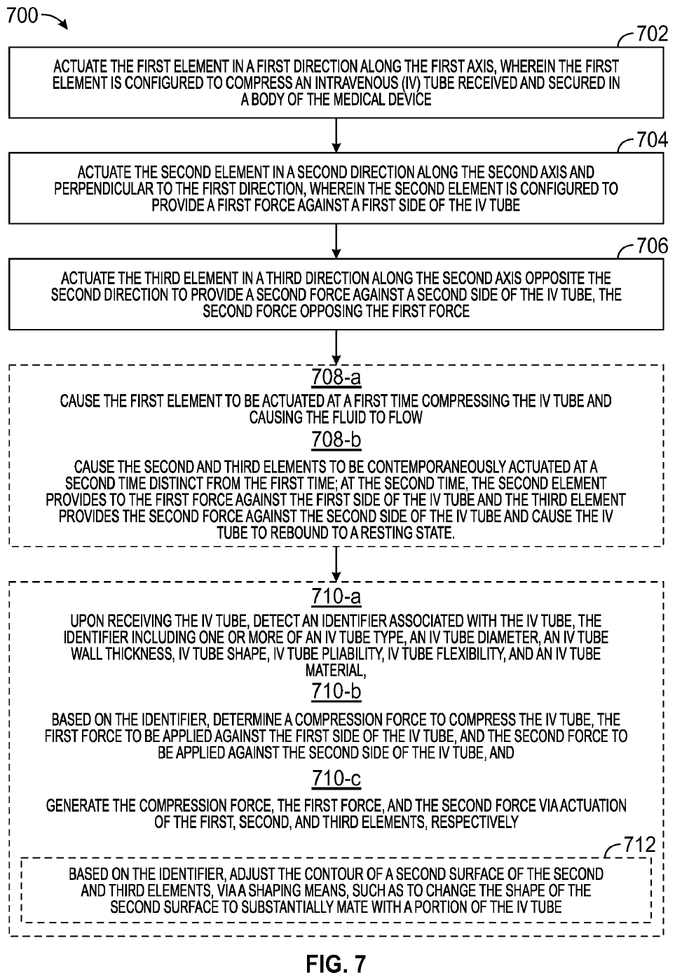

FIG. 7 illustrates a flow diagram of a method for controlling a flow rate of a medica device, in accordance with some implementations. Method 700 can be performed at a medical device described above in reference to FIGS. 1 and 2. In particular, the method can be performed at a medical device including a body and a pump mechanism. The body is configured to receive and secure an IV tube in an extended position. The IV tube is configured to allow for the transportation of a fluid. As described above in reference to FIG. 4A-5C, in some implementations, the pump mechanism includes a first element positioned along a first axis, a second element positioned along a second axis perpendicular to the first axis, and a third element positioned along the second axis opposite the second element. At least some of the operations shown in FIG. 7 correspond to instructions stored in a computer memory or computer-readable storage medium (e.g., storage, ram, and/or memory 61; FIG. 1). Operations 702-708 can also be performed in part using one or more processors and/or using instructions stored in memory or computer-readable medium of an electronic device communicatively coupled to the medical device (e.g., a server or patient care system 20 (FIG. 1) can perform operations 702-708 alone or in conjunction with the one or more processors of the medical device).

The method 700 includes actuating (702) the first element in a first direction along the first axis, wherein the first element is configured to compress an intravenous (IV) tube received and secured in a body of the medical device. For example, as shown above in reference to FIGS. 5A and 5B, a first element 305 is actuated to compress the IV tube 320.

The method 700 includes actuating (704) the second element in a second direction along the second axis and perpendicular to the first direction, wherein the second element is configured to provide a first force against a first side of the IV tube. The method 700 also includes actuating (706) the third element in a third direction along the second axis opposite the second direction to provide a second force against a second side of the IV tube, the second force opposing the first force. Examples of the second and third element are provided above in reference to FIGS. 4A-5C.

In some implementations, the method 700 includes causing (708-a) the first element to be actuated at a first time compressing the IV tube and causing the fluid to flow, and causing (708-b) the second and third elements to be contemporaneously actuated at a second time distinct from the first time. At the second time, the second element provides to the first force against the first side of the IV tube and the third element provides the second force against the second side of the IV tube and cause the IV tube to rebound to a resting state.

In some implementations, the method 700 includes upon receiving the IV tube, detecting (710-a) an identifier associated with the IV tube. The identifier includes one or more of an IV tube type, an IV tube diameter, an IV tube wall thickness, IV tube shape, IV tube pliability, IV tube flexibility, and an IV tube material. In some implementations, the method 700 further includes, based on the identifier, determining (710-b) a compression force to compress the IV tube, the first force to be applied against the first side of the IV tube, and the second force to be applied against the second side of the IV tube; and generating (710-c) the compression force, the first force, and the second force via actuation of the first, second, and third elements, respectively. In some implementations, the method 700 further includes, based on the identifier, adjusting (712) the contour of a second surface of the second and third elements, via a shaping means, such as to change the shape of the second surface to substantially mate with a portion of the IV tube. The shaping means can be one or more actuators or deformable material as described above in reference to FIGS. 4A and 4B.

Many of the above-described example steps of method 700, and related features and applications, may also be controlled by software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

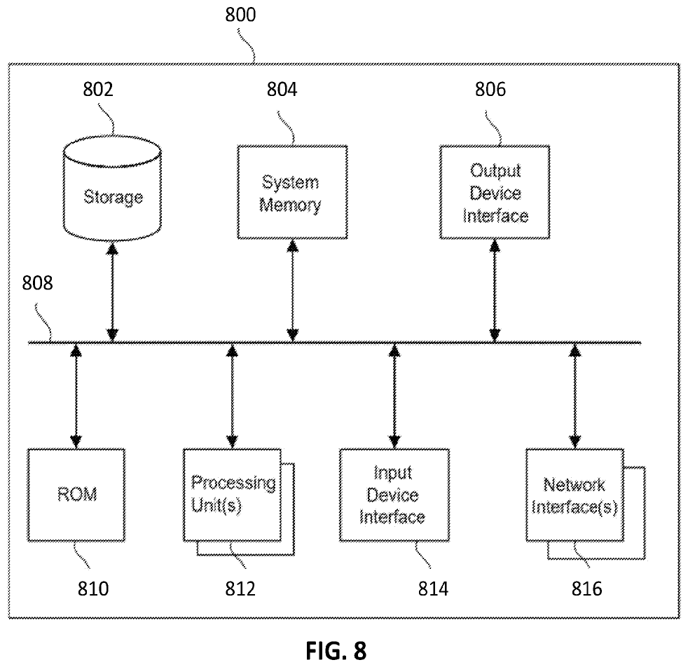

FIG. 8 is a conceptual diagram illustrating an example electronic system 800 for controlling a flow rate of a medical device, according to aspects of the subject technology. Electronic system 800 may be a specifically configured computing device for execution of software associated with one or more portions or steps of process 700, or components and processes provided by FIGS. 1 through 7, including but not limited to controller 60 of patient care system 20 and/or infusion pumps 22, 24, 26, and 28. Electronic system 800 may be representative, in combination with the disclosure regarding FIGS. 1 through 7.

Electronic system 800 may include various types of computer readable media and interfaces for various other types of computer readable media. In the depicted example, electronic system 800 includes a bus 808, processing unit(s) 812, a system memory 804, a read-only memory (ROM) 810, a permanent storage device 802, an input device interface 814, an output device interface 806, and one or more network interfaces 816. In some implementations, electronic system 800 may include or be integrated with other computing devices or circuitry for operation of the various components and processes previously described.

Bus 808 collectively represents all system, peripheral, and chipset buses that communicatively connect the numerous internal devices of electronic system 800. For instance, bus 808 communicatively connects processing unit(s) 812 with ROM 810, system memory 804, and permanent storage device 802.

From these various memory units, processing unit(s) 812 retrieves instructions to execute and data to process, in order to execute the processes of the subject disclosure. The processing unit(s) can be a single processor or a multi-core processor in different implementations.

ROM 810 stores static data and instructions that are needed by processing unit(s) 812 and other modules of the electronic system. Permanent storage device 802, on the other hand, is a read-and-write memory device. This device is a non-volatile memory unit that stores instructions and data even when electronic system 800 is off Some implementations of the subject disclosure use a mass-storage device (such as a magnetic or optical disk and its corresponding disk drive) as permanent storage device 802.

Other implementations use a removable storage device (such as a floppy disk, flash drive, and its corresponding disk drive) as permanent storage device 802. Like permanent storage device 802, system memory 804 is a read-and-write memory device. However, unlike storage device 802, system memory 804 is a volatile read-and-write memory, such as a random access memory. System memory 804 stores some of the instructions and data that the processor needs at runtime. In some implementations, the processes of the subject disclosure are stored in system memory 804, permanent storage device 802, and/or ROM 810. From these various memory units, processing unit(s) 812 retrieves instructions to execute and data to process in order to execute the processes of some implementations. Such storage and/or memory devices 802, 804 may be representative of memory 61 of controller 60.

Bus 808 also connects to input and output device interfaces 814 and 806. Input device interface 814 enables the user to communicate information and select commands to the electronic system. Input devices used with input device interface 814 include, e.g., alphanumeric keyboards and pointing devices (also called “cursor control devices”), such as those shown in controller 60 of FIG. 1. Output device interfaces 806 (e.g., shown as a display in the controller 60 of FIG. 1) enables, e.g., the display of images generated by the electronic system 800. Output devices used with output device interface 806 include, e.g., printers and display devices, such as cathode ray tubes (CRT) or liquid crystal displays (LCD). Some implementations include devices such as a touchscreen that functions as both input and output devices.

Also, as shown in FIG. 8, bus 808 also couples electronic system 800 to a network (not shown) through network interfaces 816. Network interfaces 816 may include, e.g., a wireless access point (e.g., Bluetooth or WiFi) or radio circuitry for connecting to a wireless access point. Network interfaces 816 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 800 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to specifically configured electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A medical device, comprising: a body configured to receive and secure an intravenous (IV) tube in an extended position, the IV tube configured to allow for the transportation of a fluid; a pump mechanism residing within the body, the pump mechanism including: a first element positioned along a first axis and configured to, when actuated, move in a first direction along the first axis to compress the IV tube when the IV tube is received and secured in the body, a second element positioned along a second axis perpendicular to the first axis and configured to, when actuated, move in a second direction along the second axis and perpendicular to the first direction to provide a first force against a first side of the IV tube, a third element positioned along the second axis opposite the second element and configured to, when actuated, move in a third direction along the second axis opposite the second direction to provide a second force against a second side of the IV tube, the second force opposing the first force.

Clause 2. The medical device of Clause 1, further comprising: a processor configured to: cause the first element to be actuated at a first time compressing the IV tube and causing the fluid to flow, and cause the second and third elements to be contemporaneously actuated at a second time distinct from the first time, wherein, at the second time, the second element provides to the first force against the first side of the IV tube and the third element provides the second force against the second side of the IV tube and cause the IV tube to rebound to a resting state.

Clause 3. The medical device of Clause 2, wherein the first, second, and third elements are synchronized such that the first, second, and third elements are actuated in unison with the second and third elements being actuated in directions opposite to the second and third directions, respectively, at the first time, and the first element being actuated in a direction opposite the first direction at the second time.

Clause 4. The medical device of Clause 3, wherein the first element is actuated a third time following the second time only after the IV tube is rebound to a resting configuration.

Clause 5. The medical device of any one of Clauses 1 through 4, wherein the first element comprises a first plurality of members, and each member of the first plurality of members is moved sequentially from a first end to a second end.

Clause 6. The medical device of any one of Clauses 1 through 5, wherein the second element comprises a second plurality of members and the third element comprises a third plurality of members, wherein (i) each member of the second plurality of members is moved sequentially from a first end to a second end, and (ii) each member of the third plurality of members is moved sequentially from a first end to a second end.

Clause 7. The medical device of any one of Clauses 1 through 6, wherein the pump mechanism is a first pump mechanism and the medical device further comprises a second pump mechanism, the first element is actuated by the first pump mechanism and the second and third elements are actuated by the second pump mechanism.

Clause 8. The medical device of Clause 7, wherein the body comprises a door and a housing, the door configured to couple to the housing when closed, wherein (i) the first pump mechanism is located within a part of the housing, and (ii) the second pump mechanism is located within a portion of the door.

Clause 9. The medical device of any one of Clauses 1 through 8, wherein the first element includes a first surface that compresses the IV tube, and the second and third elements have a second surface configured to reshape the IV tube.

Clause 10. The medical device of Clause 9, wherein the first surface is a flat surface configured to compresses the IV tube flat against a surface of the body, and the second surface is a concave surface configured to reshape the IV tube.

Clause 11. The medical device of Clause 9 or Clause 10, wherein the second surface includes one or more actuators, and the second surface dynamically adjusts, using the one or more actuators, to conform with the IV tube being used.

Clause 12. The medical device of Clause 11, further comprising: a processor configured to: upon receiving the IV tube, detect an identifier associated with the IV tube, the identifier including one or more of an IV tube type, an IV tube diameter, an IV tube wall thickness, IV tube shape, IV tube pliability, IV tube flexibility, and an IV tube material; and based on the identifier, adjust the contour of the second surface, via the one or more actuators, such as to change the shape of the second surface to substantially mate with a portion of the IV tube.

Clause 13. The medical device of any of Clauses 9 through 12, wherein the second surface includes deformable material, and the second surface dynamically adjusts to conform with the IV tube being used.

Clause 14. The medical device of any one of Clauses 2 through 13, wherein the processor is further configured to: upon receiving the IV tube, detect an identifier associated with the IV tube, the identifier including one or more of an IV tube type, an IV tube diameter, an IV tube wall thickness, IV tube shape, IV tube pliability, IV tube flexibility, and an IV tube material; based on the identifier, determine a compression force to compress the IV tube, the first force to be applied against the first side of the IV tube, and the second force to be applied against the second side of the IV tube; and generate the compression force, the first force, and the second force via actuation of the first, second, and third elements, respectively.

Clause 15. A method of controlling a flow rate, the method comprising: at a pump mechanism of a medical device, the pump mechanism including a first element positioned along a first axis, a second element positioned along a second axis perpendicular to the first axis, and a third element positioned along the second axis opposite the second element: actuating the first element in a first direction along the first axis, wherein the first element is configured to compress an intravenous (IV) tube received and secured in a body of the medical device; actuating the second element in a second direction along the second axis and perpendicular to the first direction, wherein the second element is configured to provide a first force against a first side of the IV tube; and actuating the third element in a third direction along the second axis opposite the second direction to provide a second force against a second side of the IV tube, the second force opposing the first force.

Clause 16. The method of Clause 15, further comprising: causing the first element to be actuated at a first time compressing the IV tube and causing a fluid to flow, and causing the second and third elements to be contemporaneously actuated at a second time distinct from the first time, wherein, at the second time, the second element provides to the first force against the first side of the IV tube and the third element provides the second force against the second side of the IV tube and cause the IV tube to rebound to a resting state.

Clause 17. The method of Clause 15 or Clause 16, further comprising: upon receiving the IV tube, detecting an identifier associated with the IV tube, the identifier including one or more of an IV tube type, an IV tube diameter, an IV tube wall thickness, IV tube shape, IV tube pliability, IV tube flexibility, and an IV tube material; based on the identifier, determining a compression force to compress the IV tube, the first force to be applied against the first side of the IV tube, and the second force to be applied against the second side of the IV tube; and generating the compression force, the first force, and the second force via actuation of the first, second, and third elements, respectively.

Clause 18. The method of Clause 17, further comprising: based on the identifier, adjusting the contour of a second surface of the second and third elements, via a shaping means, such as to change the shape of the second surface to substantially mate with a portion of the IV tube.

### Further Consideration

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The term website, as used herein, may include any aspect of a website, including one or more web pages, one or more servers used to host or store web related content, etc. Accordingly, the term website may be used interchangeably with the terms web page and server. The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

1. A medical device, comprising:
a body configured to receive and secure an intravenous (IV) tube in an extended position, the IV tube configured to allow for the transportation of a fluid;
a pump mechanism residing within the body, the pump mechanism including:
a first element positioned along a first axis and configured to, when actuated, move in a first direction along the first axis to compress the IV tube when the IV tube is received and secured in the body,
a second element positioned along a second axis perpendicular to the first axis and configured to, when actuated, move in a second direction along the second axis and perpendicular to the first direction to provide a first force against a first side of the IV tube,
a third element positioned along the second axis opposite the second element and configured to, when actuated, move in a third direction along the second axis opposite the second direction to provide a second force against a second side of the IV tube, the second force opposing the first force.

2. The medical device of claim 1, further comprising:
a processor configured to:
cause the first element to be actuated at a first time compressing the IV tube and causing the fluid to flow, and
cause the second and third elements to be contemporaneously actuated at a second time distinct from the first time, wherein, at the second time, the second element provides to the first force against the first side of the IV tube and the third element provides the second force against the second side of the IV tube and cause the IV tube to rebound to a resting state.

3. The medical device of claim 2, wherein the first, second, and third elements are synchronized such that the first, second, and third elements are actuated in unison with the second and third elements being actuated in directions opposite to the second and third directions, respectively, at the first time, and the first element being actuated in a direction opposite the first direction at the second time.

4. The medical device of claim 3, wherein the first element is actuated a third time following the second time only after the IV tube is rebound to a resting configuration.

5. The medical device of claim 1, wherein the first element comprises a first plurality of members, and each member of the first plurality of members is moved sequentially from a first end to a second end.

6. The medical device of claim 1, wherein the second element comprises a second plurality of members and the third element comprises a third plurality of members, wherein (i) each member of the second plurality of members is moved sequentially from a first end to a second end, and (ii) each member of the third plurality of members is moved sequentially from a first end to a second end.

7. The medical device of claim 1, wherein the pump mechanism is a first pump mechanism and the medical device further comprises a second pump mechanism, the first element is actuated by the first pump mechanism and the second and third elements are actuated by the second pump mechanism.

8. The medical device of claim 7, wherein the body comprises a door and a housing, the door configured to couple to the housing when closed, wherein (i) the first pump mechanism is located within a part of the housing, and (ii) the second pump mechanism is located within a portion of the door.

9. The medical device of claim 1, wherein the first element includes a first surface that compresses the IV tube, and the second and third elements have a second surface configured to reshape the IV tube.

10. The medical device of claim 9, wherein the first surface is a flat surface configured to compresses the IV tube flat against a surface of the body, and the second surface is a concave surface configured to reshape the IV tube.

11. The medical device of claim 9, wherein the second surface includes one or more actuators, and the second surface dynamically adjusts, using the one or more actuators, to conform with the IV tube being used.

12. The medical device of claim 11, further comprising:
a processor configured to:
upon receiving the IV tube, detect an identifier associated with the IV tube, the identifier including one or more of an IV tube type, an IV tube diameter, an IV tube wall thickness, IV tube shape, IV tube pliability, IV tube flexibility, and an IV tube material; and
based on the identifier, adjust the contour of the second surface, via the one or more actuators, such as to change the shape of the second surface to substantially mate with a portion of the IV tube.

13. The medical device of claim 9, wherein the second surface includes deformable material, and the second surface dynamically adjusts to conform with the IV tube being used.

14. The medical device of claim 9, wherein the processor is further configured to:
upon receiving the IV tube, detect an identifier associated with the IV tube, the identifier including one or more of an IV tube type, an IV tube diameter, an IV tube wall thickness, IV tube shape, IV tube pliability, IV tube flexibility, and an IV tube material;
based on the identifier, determine a compression force to compress the IV tube, the first force to be applied against the first side of the IV tube, and the second force to be applied against the second side of the IV tube; and
generate the compression force, the first force, and the second force via actuation of the first, second, and third elements, respectively.

15. A method of controlling a flow rate, the method comprising:
at a pump mechanism of a medical device, the pump mechanism including a first element positioned along a first axis, a second element positioned along a second axis perpendicular to the first axis, and a third element positioned along the second axis opposite the second element:
actuating the first element in a first direction along the first axis, wherein the first element is configured to compress an intravenous (IV) tube received and secured in a body of the medical device;
actuating the second element in a second direction along the second axis and perpendicular to the first direction, wherein the second element is configured to provide a first force against a first side of the IV tube; and
actuating the third element in a third direction along the second axis opposite the second direction to provide a second force against a second side of the IV tube, the second force opposing the first force.

16. The method of claim 15, further comprising:
causing the first element to be actuated at a first time compressing the IV tube and causing a fluid to flow, and
causing the second and third elements to be contemporaneously actuated at a second time distinct from the first time, wherein, at the second time, the second element provides to the first force against the first side of the IV tube and the third element provides the second force against the second side of the IV tube and cause the IV tube to rebound to a resting state.

17. The method of claim 15, further comprising:
upon receiving the IV tube, detecting an identifier associated with the IV tube, the identifier including one or more of an IV tube type, an IV tube diameter, an IV tube wall thickness, IV tube shape, IV tube pliability, IV tube flexibility, and an IV tube material;
based on the identifier, determining a compression force to compress the IV tube, the first force to be applied against the first side of the IV tube, and the second force to be applied against the second side of the IV tube; and
generating the compression force, the first force, and the second force via actuation of the first, second, and third elements, respectively.

18. The method of claim 17, further comprising:
based on the identifier, adjusting the contour of a second surface of the second and third elements, via a shaping means, such as to change the shape of the second surface to substantially mate with a portion of the IV tube.

19. A non-transitory computer readable storage medium storing one or more programs configured for execution by the one or more processors of a medical device, the one or more programs including instructions for:
at a pump mechanism of the medical device, the pump mechanism including a first element positioned along a first axis, a second element positioned along a second axis perpendicular to the first axis, and a third element positioned along the second axis opposite the second element:
actuate the first element in a first direction along the first axis, wherein the first element is configured to compress an intravenous (IV) tube received and secured in a body of the medical device;
actuate the second element in a second direction along the second axis and perpendicular to the first direction, wherein the second element is configured to provide a first force against a first side of the IV tube; and
actuate the third element in a third direction along the second axis opposite the second direction to provide a second force against a second side of the IV tube, the second force opposing the first force.

20. The non-transitory computer readable storage medium of claim 19, wherein the instructions, when executed by the one or more processors, further cause the medical device to:
cause the first element to be actuated at a first time compressing the IV tube and causing a fluid to flow, and
cause the second and third elements to be contemporaneously actuated at a second time distinct from the first time, wherein, at the second time, the second element provides to the first force against the first side of the IV tube and the third element provides the second force against the second side of the IV tube and cause the IV tube to rebound to a resting state.
